SEQUENCE LISTING (3 sequences)

SEQ ID NO: 1 - protein, 9 aa, Artificial Sequence
  Binds to oesteoblast mesenchymal stem cells
RGNXXXGGR

SEQ ID NO: 2 - protein, 12 aa, Artificial Sequence
  Binds to oesteoblast mesenchymal stem cells
VFLRGNNSGGRS

SEQ ID NO: 3 - DNA, 20 bp, Artificial Sequence
  Sequencing primer
tagcatcgtccagcgctttc

(19) United States
(12) Patent Application Publication
(10) Pub. No.: US 2011/0003702 A1
(43) Pub. Date: Jan. 6, 2011

(54) Methods, systems, and kits for identification of osteoinductive peptides

(75) Inventors: Shigemi Nagai, Lexington, MA (US); Masazzumi Nagai, Lexington, MA (US)

Correspondence Address: LAWSON & WEITZEN, LLP, 88 BLACK FALCON AVE, SUITE 345, BOSTON, MA, 02210, US

(73) Assignee: PRESIDENT AND FELLOWS OF HARVARD COLLEGE, Cambridge, MA (US)

(21) Appl. No.: 12/477,186

(22) Filed: Jun. 3, 2009

(51) Int. Cl. C40B 30/04; C07K 2/00; C40B 60/12

(52) U.S. Cl. 506/9; 530/300; 506/39

(57) ABSTRACT

Methods and systems for obtaining and identifying ligands that bind to cells and effect differentiation and/or support growth are provided herein. Several ligands were identified as proof of efficacy of the methods and systems, and amino acid sequences of compositions that effect differentiation of osteoblasts are thereby obtained.

### RELATED APPLICATION

The present application claims the benefit of U.S. provisional application Ser. No. 61/130,077, filed Jun. 3, 2008 by inventors Shigemi Nagai and Masa Nagai in the U.S. Patent and Trademark Office, and which is hereby incorporated herein by reference in its entirety.

### FIELD OF THE INVENTION

Methods and systems to screen for agents with growth or differentiation factor functions such as osteogenic activity are provided.

### BACKGROUND

Extracellular matrix (ECM) in a biological tissue is involved in recruitment, adhesion, survival, proliferation and differentiation of cells during embryonic morphogenesis, development and repair of tissues. In regenerative medicine, ECM-mimetic biomaterials play a key role in successful therapy. Design of these supportive biomaterials uses information obtained from knowledge of protein components of ECM.

ECM is composed of structural proteins found in abundant quantities, and minor amounts of specialized proteins. The structural proteins of collagens, elastins and laminins have been well characterized, however many specialized proteins remain unidentified. Moreover, detailed knowledge of functions of ECM proteins is lacking, that if known would facilitate design of ECM mimetics.

An approach is needed to obtain information regarding the role of ECM in cellular interactions such as overlaying of cells, and the role of potential peptide features appear on the surface of each ECM protein component. However even with the most advanced proteomics techniques, determining peptide sequences for protein features that are displayed on the surface of ECM proteins remains a challenge. There is a need to develop a tool to identify peptides displayed on proteins that function as ligands, and to identify corresponding ligands, for example for components of the ECM surface.

### SUMMARY

An embodiment of the invention provides a method for identifying at least one molecule having affinity for a cell receptor from a library of a plurality of molecules, the method including:

contacting cells to a screening device, the device having a supported porous mesh having a top surface and a bottom surface such that cells are contacted to the top surface, such that the pore size of the mesh retains the cells on the top surface and permits passage of nutrient media and macromolecules across the mesh (i.e., through the mesh), the device further having a bottom compartment under the supported mesh to contain a liquid in communication with the mesh; and

adding a sample of the library to the bottom portion of the device such that the library is in communication with the cells, so that the at least one molecule having affinity binds to receptors on the cell and is retained, and unbound molecules are removed, thereby identifying the at least one molecule.

In various related embodiments of the method, prior to providing the plurality of molecules, the method further includes culturing the cells. For example, the cells are cultured in contact with the top surface of the porous mesh. Alternatively, the cells are cultured separately from the porous mesh and are then transferred to the top surface of the porous mesh.

In general in various embodiments, the library comprises at least one molecule selected from the group consisting of: a peptide, a protein, a lipid, a glycan, and a small molecule chemical compound, i.e., a low molecular weight chemical.

Further, the cells are eukaryotic cells, for example human cells. In various embodiments, the cells are selected from the group of tissues of origin that include periodontal, ocular, epithelial, nerve, scalp (hair follicle), and endocrine. In various embodiments of the method the cells are stem cells, for example, mesenchymal stem cells, for example, the stem cells are derived from osteoblast cells.

An embodiment of the method further includes identifying the molecule bound to the receptor by at least one technique selected from the group consisting of: mass spectrometry, flow cytometry, and optical photometry. These techniques are suitable for any of the classes of molecules. In general, the molecule is a peptide. In an embodiment of this method, the peptide is provided as a recombinant fusion to a bacteriophage coat protein, the library is a phage display library, and the method further involves contacting the eukaryotic cell, after growth on the mesh to form a confluent layer of cells, with a sample of the phage display library. Contacting the eukaryotic cells with the phage involves inverting the insert having the supported mesh, i.e., rotating the insert so that cells retained on the mesh, previously on the top are now on the bottom, and are in communication with library because the mesh is sufficiently porous to allow communication and contact of the phage and eukaryotic cells.

In the embodiment of the methods herein in which the library is a phage display library, bacteria display the phage library as at least a portion of the phage remain attached to phage-producing bacterial cells. Alternatively, the phage library is free of bacterial cells. The method in various embodiments further involves screening the at least one molecule that is a peptide by iterative cycles of affinity selection and bacteriophage amplification, to enrich for those phage clones having desired properties of binding to the target and stimulating the cells. The method in various embodiments further includes identifying peptide capable of binding to the receptor by obtaining a nucleotide sequence of at least one recombinant fusion gene that encodes the peptide. The peptide in various exemplary embodiments of the method identified by these methods has affinity for an extracellular matrix (ECM) receptor. It is envisioned herein that the methods and systems are suitable for a variety of additional extracellular targets and cell-bound receptors.

In a related embodiment, the method further includes producing the peptide by expressing the recombinant fusion gene in the bacterial cells. For example, producing the peptide further includes isolating the phage carrying the peptide fusion on an agar plate (i.e., obtaining bacterial colonies bearing phage, or phage plaques, using agar-based solid media such as nutrient medium), for example for additional screening. Alternatively, producing the peptide involves synthesizing the predicted amino acid sequence chemically on a peptide synthesizer using the nucleotide sequence. In various embodiments, the method further involves demonstrating that the peptide stimulates osteoblast differentiation.

Another embodiment of the present invention provides a peptide identified by any of the above methods.

Another embodiment of the present invention provides a system for biopanning including: a culture container and a culture insert for the culture container, the insert having sides and a porous mesh supported in a plane substantially perpendicular to the sides of the insert and container, and parallel to the bottom of the container. In one embodiment, the porous mesh divides the insert into at least one chamber, and alternatively a commercially available insert having two chambers can be used. The mesh has a pore size sufficiently small to retain a eukaryotic cell and sufficiently large to permit passage of bacteriophage and bacterial cells. The mesh surface is suitable for adhesion of the cell and consequently for growth of the cell. The insert has an outer diameter less than an inner diameter of the cell culture container and a cross-sectional shape smaller than the inner shape of the cell culture, for example, the cross-sectional shape of the insert is congruent to that of the cell culture container. Following deposition of cells on the mesh and adhesion and growth of the cell, the insert is removed from the culture container, and is inverted into a medium containing a sample of a phage library, the cells adhering to the mesh in an inverted position, and contacting members of the phage library.

In general for retention of the eukaryotic cells and communication of the phage library for contact with the cells, the pore size of the mesh is selected from the group of: about 2 μm, about 5 μm, about 10 μm, about 20 μm, about 50 μm, and about 100 μm. In general, the system is sterilizable, and the porous mesh is located on an insert that is insertable and removable with respect to the culture container and consequently is invertible.

In various embodiments, the system provides a platform for cells, for example, cultured cells, but also suitable for primary cells, contacted to porous mesh for adhesion and growth, on a platform in an insert having at least one chamber for growth. In an embodiment of the system in which the insert has a single chamber during the growth of cells, the insert is removed from the culture container, is inverted, and is circumferentially wrapped around the outer diameter of the insert to the mesh with Parafilm, such that the Parafilm wrapping forms a chamber above the inverted mesh platform. The height of Parafilm wrapping extends beyond the length of the insert and forms a chamber which is a container and/or well for addition of the phage library.

Also provided herein is a kit for identifying a molecule of interest, the kit comprising the system for biopanning according to methods herein, a container for the kit, and instructions for use. The kit optionally includes at least one of a culture container, a culture insert, a phage fusion library having a plurality of peptide amino acid sequences, a phage fusion which is a negative control, and a phage fusion which is a positive control.

Another embodiment of the present invention provides a peptide composition having amino acid sequence RGNxxxGGR (SEQ ID NO: 1), such that RGN and GGR are amino acids indicated by the one letter code, and x is any amino acid. The peptide composition in one embodiment has an x such that the amino acid is naturally occurring. Alternatively, x is non-naturally occurring and synthetic. In another embodiment, x is a linker comprising a non-peptide component. For example, x is a peptide nucleic acid. In a related embodiment, the amino acid sequence further includes VFLRGNNSGGRS (SEQ ID NO: 2).

In an exemplary embodiment, the peptide composition stimulates growth or differentiation of a cell. For example, the cell is a eukaryotic cell, for example, a human cell. In various embodiments the cell is a stem cell, for example, a mesenchymal stem cell. In various embodiments of the kit, the stem cells are derived from osteoblast cells.

### BRIEF DESCRIPTION OF THE DRAWINGS

FIG. 1 is a drawing of a biopanning chamber and culture of osteogenic target cells on a porous membrane having a pore size of 8 μm.

FIG. 1 panel A is a drawing of a porous membrane or mesh, 101, supported on internal surfaces of the insert, 102. The mesh has a pore size of 8 μm that was used to maintain target cells and permit free passage of the library of molecules to access the target cells.

FIG. 1 panel B is a drawing showing the membrane or mesh, 101, located transversely across a stage or platform within a cylindrical insert, 102, that has an outer diameter narrower that a culture container, 103. Osteogenic target cells, 104, were inoculated onto the top surface of the membrane on the insert, and the insert was placed in a sterile culture container with an appropriate cell culture medium, 105, until reaching cell confluence was obtained.

FIG. 1 panel C shows the insert, 102, that was inverted at appropriate cell density, in the culture container, 103, containing cell culture medium, 105. The osteogenic target cells, 104, following growth to confluence within the insert adhered to the membrane, 101, and was inverted for addition of the peptide library. The cells following inversion were located on the downward facing or lower surface of the insert mesh.

FIG. 1 panel D shows addition of peptide-displaying bacterial phages, 106, to the membrane, 101, within the cylindrical insert, 102, and binding of the peptide-displaying bacterial phages to the osteogenic target cells, 104. The insert is located in the container, 103, containing the culture medium, 105. After binding, peptide-display bacterial phages having amino acid sequences capable of binding to target cells were selected and enriched and positive clones are isolated on agar plates.

FIG. 2 is a drawing showing systems and methods for biopanning to obtain peptide-displaying phage clones that bind to osteogenic cells.

FIG. 2 panel A shows a culture container in which sterile osteogenic basal medium, 201 (Lonza, Hopkinton Mass.) was placed in the lower compartment, 202, and osteogenic cells, 203, were grown to confluence on the membrane or mesh, 101, located transversely within the cylindrical insert, 102.

FIG. 2 panel B shows inversion of the insert, 102, with osteogenic cells, 203, on the membrane, 101.

FIG. 2 panel C shows the inverted insert, 102, wrapped with Parafilm, 204, around the side of the insert, or wrapped with another flexible material, to form a chamber or well having the membrane, 101 as a floor or bottom of the newly formed chamber.

FIG. 2 panel D shows a peptide-displaying bacterial phage library, 106, added to the chamber formed by the Parafilm, 204, above the membrane, 101, within the insert, 102. Phage clones of the library bearing peptides of amino acid sequences having affinity for receptors on the osteogenic target cells, 203 were bound to the cells on the mesh. The insert was inverted and placed into a fresh culture container having appropriate buffer or medium, 201.

### DETAILED DESCRIPTION

The current paucity of information available regarding the structure of ECM protein receptors is a major barrier to finding methods for searching for potential ligands of these receptors. The structure of ligands is unknown, and these ligands are postulated to have functional ability to bind specifically to ECM receptors of cells, which ECM receptors also remain uncharacterized. Hence the field faces barriers to development of lead compounds for potential therapeutic agents.

An approach provided herein to address the problem of unknown peptide ligands is to screen a peptide library using in vivo ECM receptors located on living cells as a target, or “bait”. Phage display peptide libraries are commercially available, for example from Dyax Corp. (Cambridge, Mass.) and from Invitrogen (San Diego, Calif.).

An exemplary peptide library is composed of random peptides dodecamer members (12 mer), and about 200 million species includes only a portion of the set of all possible sequences. The theoretical total number of species is calculated as 20 (naturally occurring amino acids) to the 12^{th} power. This 200 million member portion of the library is envisioned to include at least some functional peptides that mimic ECM peptide ligands based on consideration of physicochemical similarities of amino acids. As practical information regarding the nature of proteins involved in ECM receptor structure in vivo is also lacking, the methods herein use as a target in vivo cells, thus the plasma membrane with ECM receptors of a cell is bait for identifying suitable peptide ligands.

Methods herein are used to obtain peptides, to facilitate analyzing interactions between peptide ligands and receptors or other sites on the plasma membrane ECM. Methods herein involve “biopanning”, a procedure that has been found useful to isolate peptides possessing affinity for an identified protein, however with live eukarytic cells in culture as the target.

Previously described biopanning methods generally have used as a target an immobilized protein, usually a purified immobilized protein, which as it is removed from the in vivo context has lost functional three-dimensional structural and functional characteristics of the in vivo protein. This limitation is particularly pertinent for transmembrane proteins such as ECM receptor proteins. Membrane proteins in vivo have a natural polarity that includes a set of surfaces directed into the cell and another set of surfaces that are exposed to the extracellular environment, which are generally hydrophilic. Membrane proteins further include highly hydrophobic transmembrane regions. Under conditions in which the protein is purified, these hydrophobic regions are insoluble and form micelles and artificially adhere to irrelevant surfaces such as glassware and plasticware. These properties of transmembrane proteins result reduced availability of sites for identifying and binding of potential ECM-mimetics. The methods and systems in Examples herein were developed to surmount these problems.

Systems herein include culture inserts having a transverse membrane or mesh. These inserts, while commercially available in cylindrical form, can also be manufactured in any geometrical shape convenient to a culture container and are not limited by cross-sectional geometry. In general, the inserts are sterile, and sterilizable, for example by conventional one or more methods such as autoclaving, boiling, radiation and exposure to sterilizing gases. The methods of manipulation herein include conventional microbiological and cell culture standards, such as in a sterile environment, for example, a sterile hood, or a sterile glove box etc. The inserts used in systems herein are consequently manufactured from materials suitable for autoclaving such as glass or plastics, e.g., polycarbonate, polyethylene, isoprene, Teflon, etc. In various embodiments, at least one of the inserts and culture containers have sterile covers, although the inserts once placed inside the culture container with a cover need not be independently covered.

The membrane useful for the systems and methods herein can be manufactured from any material which has a suitable pore size, and has surface properties that support adhesion and growth of eukaryotic cells. The pore size is sufficiently large to permit passage of bacteria, bacteriophage, and macromolecules. In general, the insert is insertable and removable, and consequently invertible. An exemplary suitable membrane is manufactured from a suitable synthetic organic polymer material, for example hydrophilic polytetrafluoroethylene (PTFE), cellulose ester(s), polycarbonate, polyethylene, terepthalate, cellulose acetate etc. The membrane under certain circumstances for growth of particularly fastidious cells is pre-treated, i.e., rinsed to remove unwanted inorganic and/or organic substances from the membrane that may have been added to preserve or stabilize the membrane. Pretreating the membrane to remove such substances can improve eukaryotic cell viability, adhesion, and growth, and reduce non-specific binding of phage and bacterial cells to the membrane.

The invention having now been fully described, a skilled person will recognize that many suitable designs may be substituted for or used in addition to the configurations described above. It should be understood that the implementation of other variations and modifications of the embodiments of the invention and its various aspects will be apparent to one skilled in the art of cell biology and molecular biology, and that the invention is not limited by the specific embodiments described herein and in the claims. Therefore, it is contemplated that modifications, variations, or equivalents fall within the true spirit and scope of the basic underlying principles disclosed and claimed herein. The contents of literature cited herein are hereby incorporated herein by reference in their entireties.

### EXAMPLES

### Example 1

### Design of a Biopanning Chamber

A biopanning apparatus system was designed for biopanning in which target membrane protein (“bait”) is maintained in a physiological environment and in a configuration and structure of the cell in vivo.

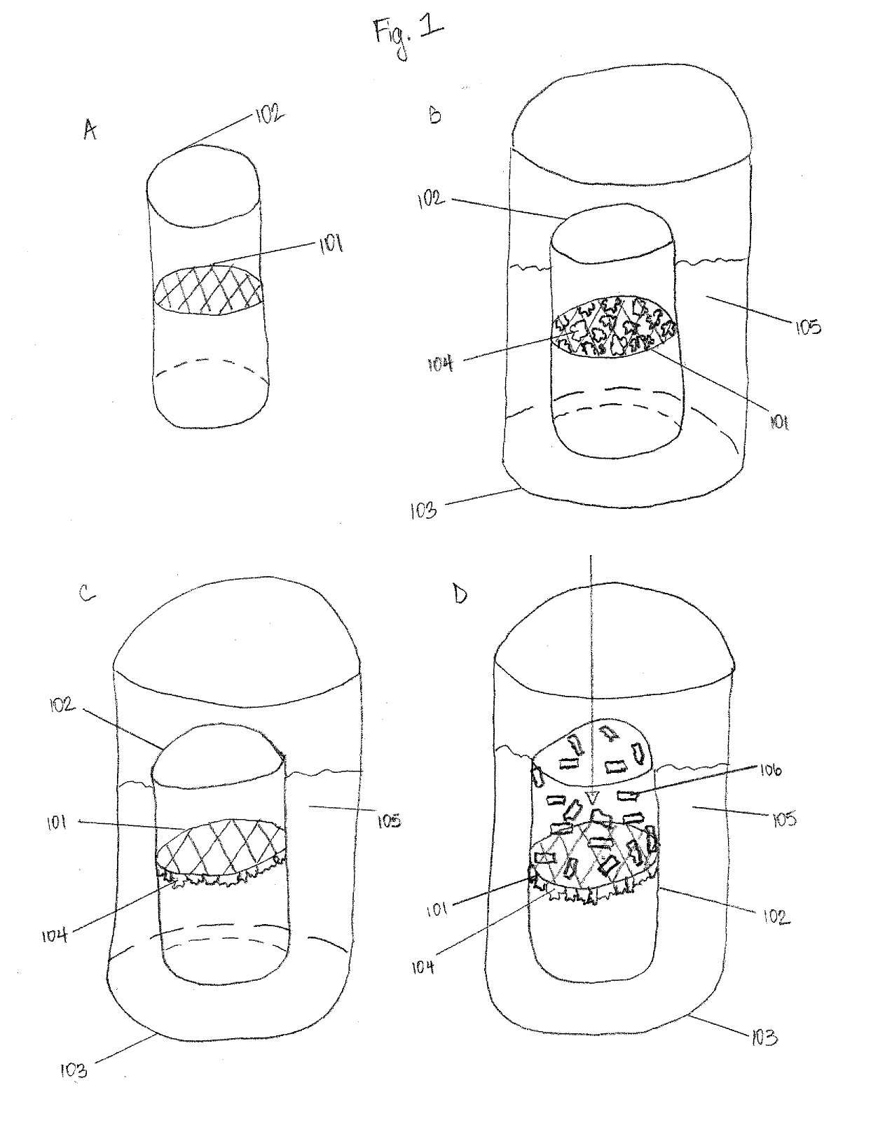

The biopanning system used herein includes a cell culture insert having upper and lower compartments separated by a platform which is or includes a mesh filter having about 5 μm to about 10 μm pore size. See FIG. 1.

Target ECM cells were grown to confluence on a mesh filter in the upper chamber of the insert, in which the insert was placed into a culture container. The growth resulted in confluent cells that adhered to the surface of the mesh, an indication that under these circumstances the cultured ECM cells exhibited normal eukaryotic cell properties.

Culture inserts having two chambers, with suitable pore-sized meshes separating the chambers, are commercially available in a variety of pore sizes and diameters from Millipore (Single-well Millicell Inserts and Multiwell Minicell Inserts, Millipore Corp., Billerica Mass.), and from BD Falcon (Cell Culture Inserts and Multiwell Insert System, BD Falcon, Franklin Lakes, N.J.).

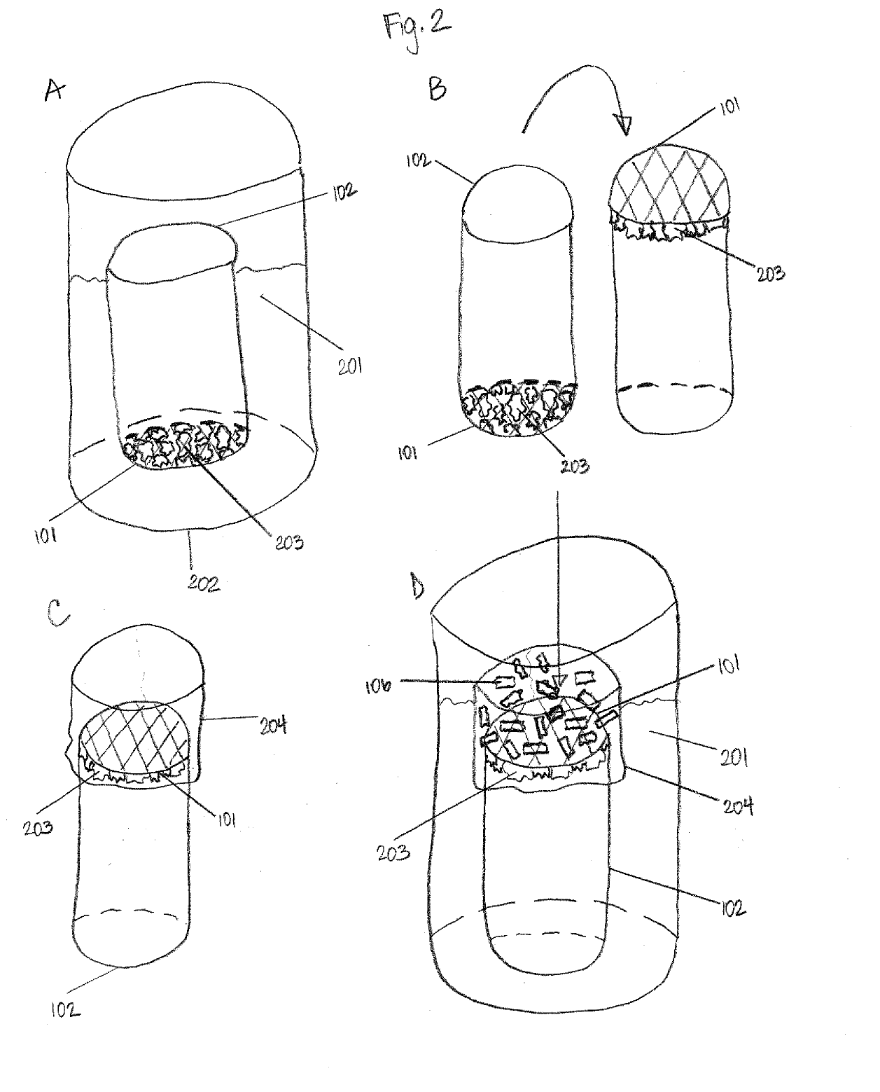

The cell culture insert alternatively used herein has a single chamber or compartment, and the porous mesh is located at a bottom or floor of the compartment, parallel and in contact with the bottom or floor of the outer culture container. In this configuration, the single chamber was formed from a cylinder having one closed end and one open end, the closed end being the mesh, and the insert with one chamber inserted into the culture container and filled with medium. Cells were grown on the mesh in appropriate cell culture medium supplied above the cells and in the outer container. See FIG. 2. After suitable cell adherence of the cells to the mesh and cell growth, the insert was removed, and inverted in a position that the mesh forms a top or ceiling. Parafilm was wrapped around the top or ceiling of the insert extending above the horizontal level of the porous mesh, forming an additional chamber or bio-panning well. A sample of a phage library was added to this well as shown in examples herein.

### Example 2

### Mesenchymal Cells from Human Bone Marrow

Mesenchymal stem cells were derived from human bone marrow, hMSC, and were obtained commercially for use as target cells (Lonza; Allendale, N.J.). hMSC cells were seeded in a cell culture insert having a culture surface was made of PET (polyethylene terephthalate)-mesh filter with 8-μm pore size and 0.3-cm² cell growth area (BD Falcon; Franklin Lakes, N.J.). Eighty-thousand cells were seeded and were grown in osteogenic basal medium (Lonza; Allendale, N.J.) supplemented with 10% FBS and were incubated for 3 days to form confluent monolayer.

### Example 3

### Peptide Display Bacterial Phages

One vial of a bacterial culture carrying peptide display phages (Flitrx peptide display library, Invitrogen, containing 2×10¹⁰ cfu/ml) was inoculated into 50 ml of tryptophan-free IMC medium (Flitrx panning kit, Invitrogen; Carlsbad, Calif.) containing 100 μg/ml of ampicillin (IMC^{amp}). Bacterial cells were incubated overnight at 25° C. with shaking at 250 rpm. After overnight growth, 10¹⁰ bacterial cells were resuspended in 50 ml fresh IMC^{amp} supplemented with 100 μg/ml of tryptophan to induce peptide, and bacterial cells were grown at 25° C. with shaking for 6 h for induction of expression of peptide.

### Example 4

### Biopanning of Peptide Phages with Mesenchymal Cells

Bacterial cells carrying peptide displaying phages were resuspended at 10⁹ cells/ml in 50 mM Hepes buffer, pH 7.5, with 1% nonfat dry milk, 146 mM NaCl, 4 mM KCl, 1 mM CaCl₂, 0.5 mM MgCl₂, 5 mM glucose and 1% α-methylmannoside. Cell concentration was determined by optical density (OD) at 600 nm.

The culture insert was inverted as described herein, and was encircled and/or fenced with a Parafilm “chimney” to form a culture space above the mesh which forms a bottom of the space or chamber, the mesenchymal cells adhering to the lower surface of the mesh (FIG. 2).

Two hundred microliters of bacterial cell suspension having 2×10⁸ bacterial cells (one copy of each clone) were incubated with hMSC cells for 5 min with gentle rocking to select for phage particles displaying amino acid sequences capable of binding to the hMSC cells.

The mesh surface was washed with the suspension buffer three times remove unbound phage. Phage clones bound to the hMSC cells were obtained by vortexing in IMC^{amp}, and clones were expanded by growth overnight at 25° C. with shaking at 250 rpm.

Serial dilutions of resulting selected phage were prepared and were spread onto the surfaces of RMG ampicillin agar plates (Flitrx panning kit; Invitrogen) to obtain appropriate numbers of bacterial clone colonies. Ninety-five bacterial colony clones were picked and transferred into 100 μl of IMC medium in a 96-well plate. The parental strain of non-recombinant bacterial phage was also inoculated to a control well or plate. Phage clones were expanded by growth in bacterial medium overnight. Expression of the peptides in each phage clone was induced by addition of tryptophan and growth for 6 h.

### Example 5

### Secondary and Tertiary Screening of Selected ECM Receptor-Affinity Phage Clones

The processes by which osteoblast progenitor cells differentiate into mature osteoblasts to produce bone include: migration to bone surface, with bone ECM proteins having active sequences exposed due to osteoclast activity; differentiation into osteoblasts and replication; and expression of osteoblast marker genes, such as type I collagen and alkaline phosphatase, resulting from signaling by a component of ECM.

A secondary screen of clones bearing peptides was designed to determine activity of these peptides by a parameter chosen to measure growth of mesenchymal stem cells, since cell replication is an early function of osteoblast progenitor cells. For example a parameter measures cell content of ATP, respiration, DNA synthesis, or other physiological measure of active cell growth.

A convenient parameter chosen herein was to assay cell ATP content using commercially available the CellTiter Glo ATPase assay (ProMega; Madison, Wis.). This assay is capable of detecting as few as 50 cells, and the assay involves adding a reagent directly into the cell culture and determining luminescence (homogeneous assay mode).

For a tertiary screen of the peptides identified, the effect of each peptide on target cells was determined by measuring resulting amounts of the osteoblastic marker alkaline phosphatase. Each of these procedures is appropriate for methods using clonally purified individual isolates, or sibling pools.

Phage clones were induced to express peptides, and were heat-devitalized in boiled water, viz., to eliminate viable bacteria. Fifty microliters of hot phage suspension was poured into a 96-well plate containing agar, which was centrifuged at 2000 rpm to embed phages in the surface of the agar. As hMSC cells do not replicate on agar, therefore hMSC cell metabolism such as measured by ATP content increases only when contacted by a selected peptide capable of contacting an ECM receptor and consequently stimulating and supporting the growth of the cells.

hMSC cells were seeded at 20,000 cells/well onto phage-embedded agar in osteogenic basal medium with 10% FBS. Cell growth was evaluated at 72 h using a commercially available kit, the CellTiter-Glo™ Luminescent Cell Viability Assay (Promega; Madison, Wis.).

Phage clones that supported hMSC cell growth were further evaluated for induction of an enzyme marker of osteoblastic differentiation, alkaline phosphatase (AP). AP activity was measured by Sensolyte AP assay kit (Anaspec; San Jose, Calif.).

### Example 6

### Identification of Osteoinductive Peptide Sequence

Selected phage clones were expanded and DNA was isolated by the following procedures. Each clone was grown in 100 μg/ml ampicillin containing RM medium (Flitrx kit, Invitrogen) at 30 C overnight. Phage DNA was isolated with S.N.A.P. Miniprep kit (Invitrogen; Carlsbad, Calif.) for DNA sequencing, to determine predicted amino acid sequences of peptide displayed on each clone. DNA was sequenced using a reverse sequencing primer: TAGCATCGTCCAGCGCTTTC (SEQ ID NO: 3; Seqwright, Houston Tex.).

### Example 7

### Selection of Specific Phage with Affinity for ECM

Peptide displaying phages were inoculated as described above into an inverted insert with a chamber having eukaryotic cells located on the opposite side of the mesh in comparison to the side used for growth of these cells. Potential peptide ligands on the phage surface had access to contact ECM receptors on the eukaryotic cell basal plasma membrane by permeation into and across the pores of the mesh. The resulting affinity-selected peptide clones were isolated on an agar plate for secondary screening for presence of an activity causing specialized cell differentiation as a result of binding of the peptide. Differentiation inductive clones were identified, and nucleotide sequences determined to obtain the amino acid sequences of the selected peptides.

### Example 8

### Support of Growth of Osteoblastic Precursor Cell Differentiation by Selected Phage Clones

Peptides were sought by the methods herein that would support osteoblastic differentiation from mesenchymal stem cells. An initial biopanning experiment was performed with a two-chamber biopanning insert that was modified from a commercially available single-chamber filter insert with Parafilm as shown in FIGS. 1 and 2. The single chamber insert has a 25-mm chamber diameter and 8 μm-filter pore. Six phage clones were isolated from 96 phage clones obtained from the screen, and these clones were observed to support growth of osteoblastic precursor cells.

The secondary and tertiary screening included inoculating positive clones into a 96-well culture plate and growing the clones overnight. The culture plates were sealed and bacteria were killed by incubating the plates at 100° C. The heat devitalized phage preparations free of viable bacteria were inoculated and embedded into a 96-well agar plate. Aliquots of each clone were preserved as glycerol-stocks at 80° C.

Osteoblast progenitor cells were seeded into the agar plates having the devitalized bacteria. The growth of the osteoblast cells was observed by measuring amount of cellular ATP after an appropriate incubation. The data observed are shown in Table 1. Bacterial clones bearing phage that stimulate osteoblast precursor cells were selected from observations of cells having the highest relative ATP luminescence units, and these clones were further characterized.

### Example 9

### Identification of Peptide Sequences

Peptides were further tested to determine ability to stimulate activity of osteoblastic marker enzyme alkaline phosphatase (AP). Results shown in Table 1 indicate that several clones induced an order of magnitude more AP expression than the control empty vector phage.

DNA from a phage clone expressing a peptide that stimulated AP expression and was isolated and was sequenced. The amino acid sequence obtained, using the one letter code was VFLRGNNSGGRS (SEQ ID NO: 2). This sequence was not found in any of the databases that were searched, indicated that the sequence was not previously been reported.

Analysis of the sequence revealed presence of tripeptides RGN and GGR, which are consensus tripeptides previously associated with osteoinduction. Isolation of a phage clone encoding this dodecamer sequence demonstrates successful proof of concept and reduction to practice of the methods herein. The isolated dodecamer sequence is potentially a more potent osteogenic peptide than either of the tripeptide sequences RGN and GGR.

It is envisioned herein that that the methods and apparatus provided can identify multiple novel peptide ligands with more potent growth and/or differentiation stimulating abilities, such as osteoinductive activity, using any eukaryotic cell types.

The biopanning methods described herein are simple and practical. The bait and/or target proteins resulting from use of the system for biopanning, as shown herein for the ECM proteins, were maintained as native three-dimensional structures during the process of binding peptide. Proteins present on living cells provide authentic in vivo signals as biopanning targets. Methods herein are suitable for isolation of peptides that would serve as lead compounds to develop therapeutic agents to treat a variety of conditions, and such peptides would thereby facilitate drug development. Furthermore, the biopanning procedure and system is applicable to a variety of types of adherent cells that construct various tissues such as periodontal tissues, eye lens, nerve, hair follicles, endocrine tissues, etc. The methods and systems are applicable to screening and identifying surface ligands that mediate not only cell-to-cell adhesion, but interconnection of various vertebrate systems, as well as maintenance of tissue integration, wound healing, cellular migration, and metastasis.

Moreover, the probing target need not be limited to peptides. For example, small molecules, proteins, lipids, and glycans are each capable of traversing the mesh, and thus can also be analyzed in this biopanning method.

TABLE 1
Cellular ATP from relative luminescence per cell number in cells contacted with clones of devitalized phage

| | 1 | 2 | 3 | 4 | 5 | 6 | 7 | 8 | 9 | 10 | 11 | 12 |
|---|---|---|---|---|---|---|---|---|---|---|---|---|
| A | 7,075 | 3,335 | 3,550 | 2,955 | 3,765 | 3,880 | 3,715 | 5,935 | 3,240 | 2,415 | 4,630 | 4,050 |
| B | 9,245 | 7,300 | 4,160 | 2,380 | 3,035 | 3,530 | 2,800 | 3,470 | 2,325 | 2,820 | 3,360 | 2,930 |
| C | 3,105 | 2,505 | 5,255 | 2,885 | 3,455 | 2,905 | 4,140 | 2,540 | 2,425 | 2,140 | 2,270 | 2,590 |
| D | 5,425 | 3,740 | 16,430 | 4,135 | 3,835 | 3,865 | 3,380 | 3,650 | 3,775 | 4,160 | 3,555 | 6,195 |
| E | 3,495 | 4,870 | 18,270 | 6,205 | 6,710 | 5,385 | 3,600 | 5,605 | 6,040 | 5,785 | 5,655 | 5,560 |
| F | 5,540 | 7,790 | 7,780 | 8,760 | 8,550 | 6,145 | 4,530 | 5,290 | 8,410 | 40,665 | 16,200 | 6,480 |
| G | 8,335 | 10,945 | 75,455 | 72,525 | 41,385 | 9,055 | 5,965 | 6,810 | 8,070 | 11,615 | 7,905 | 26,700 |
| H | 4,320 | 6,199 | 8,356 | 3,870 | 9,063 | 8,993 | 8,890 | 48,890 | 39,150 | 8,745 | 39,530 | 8,570 |

Position A1: empty vector control clone
Positions: Clones at positions F10, G3, G4, G5 and H8 showed significantly greater luminescence than the control, yielding 5.8, 10.7, 10.3, 5.9, and 6.9, respectively, fold more ATP per cell than the control.

What is claimed is:

1. A method for identifying at least one molecule having affinity for a cell receptor from a library of a plurality of molecules, the method comprising:
contacting cells to a screening device, the device comprising a supported porous mesh having a top surface and a bottom surface wherein cells are contacted to the top surface, wherein pore size of the mesh retains the cells on one surface and permits passage of nutrient media and macromolecules across the mesh, the device further having a bottom compartment under the supported mesh to contain a liquid in communication with the mesh; and
adding a sample of the library to the bottom portion of the device in communication with the cells, whereby the at least one molecule having affinity binds to receptors on the cell and is retained, and unbound molecules are removed, thereby identifying the at least one molecule.

2. The method according to claim 1, wherein prior to providing the plurality of molecules, the method further comprises culturing the cells.

3. The method according to claim 2, wherein the cells are cultured in contact with the top surface of the porous mesh.

4. The method according to claim 2, wherein the cells are cultured separately from the porous mesh and are then transferred to the porous mesh.

5. The method according to claim 1, wherein the library comprises at least one molecule selected from the group consisting of: a peptide, a protein, a lipid, a glycan, and a small molecule chemical compound.

6. The method according to claim 1, wherein the cells are eukaryotic.

7. The method according to claim 6, wherein the cells are of human origin.

8. The method according to claim 6, wherein the cells are derived from at least one tissue selected from the group consisting of: periodontal, ocular, epithelial, nerve, hair, and endocrine.

9. The method according to claim 1, wherein the cells are stem cells.

10. The method according to claim 9, wherein the stem cells are mesenchymal stem cells.

11. The method according to claim 1, further comprising identifying the molecule bound to the receptor by at least one technique selected from the group consisting of: mass spectrometry, flow cytometry, and optical photometry.

12. The method according to claim 1, wherein the molecule is a peptide.

13. The method according to claim 12, wherein the peptide further is a recombinant fusion to a bacteriophage coat protein, the library is a phage display library, and the method further comprises contacting the eukaryotic cell with a sample of the phage display library.

14. The method according to claim 13, wherein contacting the eukaryotic cell with the phage further comprises inverting the supported mesh wherein cells retained on the mesh are in communication with library.

15. The method according to claim 13, wherein the phage display library is a library of bacteria displaying the phage display library attached to phage-producing bacterial cells.

16. The method according to claim 14, further comprising identifying the peptide by obtaining a nucleotide sequence of at least one recombinant fusion gene encoding the peptide bound to the receptor.

17. The method according to claim 16, further comprising producing the peptide by expressing the recombinant fusion gene in the bacterial cells.

18. The method according to claim 16, wherein the peptide has affinity for an extracellular matrix (ECM) receptor.

19. The method according to claim 17, wherein producing the peptide further comprises isolating the phage carrying the peptide fusion on a solid nutrient medium for additional screening.

20. The method according to claim 16, wherein producing the peptide further comprises synthesizing on a peptide synthesizer the peptide obtained from the nucleotide sequence.

21. The method according to claim 13, wherein the at least one molecule that is a peptide is screened by iterative cycles of affinity selection and bacteriophage amplification.

22. The method according to claim 12, wherein the peptide stimulates osteoblast differentiation.

23. A peptide identified by the method according to claim 1.

24. A system for biopanning comprising:
a culture insert having sides and a porous mesh supported in a plane substantially parallel to the bottom of the container, the porous mesh dividing the insert at least one chamber, wherein the mesh has a pore size sufficiently small to retain a eukaryotic cell and sufficiently large to permit passage of bacteriophage, wherein the mesh further provides a surface for adhesion and growth of the cell, the insert having an outer diameter less than an inner diameter of a cell culture container and a circumference congruent to the cell culture container, wherein following growth of the cell, the insert is inverted into a phage library, wherein cells adhere to the mesh in an inverted position, and contact members of the phage library.

25. The system according to claim 24, wherein the system is sterilizable.

26. The system according to claim 24, wherein the porous mesh is insertable and removable.

27. The system according to claim 24, wherein the porous mesh is invertible.

28. The system according to claim 24, further comprising cultured cells contacted to the porous mesh.

29. The system according to claim 24, further comprising prior to inverting the insert into a phage library, wrapping the outer diameter of the insert parallel to the mesh with Parafilm, wherein the Parafilm wrapped around the insert extends beyond the outer diameter of the insert parallel to the mesh creates a container for the phage library.

30. A kit for identifying a molecule of interest, the kit comprising the biopanning system according to claim 24, a container and instructions for use.

31. The kit according to claim 30, further comprising a library having a plurality of molecules.

32. The kit according to claim 30, wherein the molecules are bacteriophages.
